(19) United States
(12) Patent Application Publication
(10) Pub. No.: US 2021/0038760 A1
(43) Pub. Date: Feb. 11, 2021

(54) BIOMIMETIC BONE COMPOSITE MATERIAL, A PREPARATION METHOD AND USES THEREOF

(71) Applicant: SICHUAN UNIVERSITY, Chengdu (CN)

(72) Inventors: Fang WU, Chengdu (CN); Ruijuan YAO, Chengdu (CN); Guolong MENG, Chengdu (CN); Bo ZHANG, Chengdu (CN); Jing HE, Chengdu (CN); Juan LIU, Chengdu (CN)

(21) Appl. No.: 16/986,390

(22) Filed: Aug. 6, 2020

(30) Foreign Application Priority Data
Aug. 7, 2019 (CN) 201910725714.1

(51) Int. Cl. A61L 27/02; A61L 27/22; A61L 27/24; A61L 27/12; A61L 27/44

(52) U.S. Cl. CPC A61L 27/025; A61L 27/222; D04H 1/728; A61L 27/12; A61L 27/446; A61L 27/24

(57) ABSTRACT

The present invention relates to the field of functional materials for medical use, and particularly to a biomimetic bone composite material, a preparation method and uses thereof. The biomimetic bone composite material provided by the invention is prepared from the raw material containing the following components: gelatin and/or collagen, hydroxyapatite and a silicon source. The present invention also provides a preparation method of the composite material and uses thereof in preparing bone repair materials. The highly biomimetic composite material with fibrous network structure prepared by the present invention can simulate the microenvironment similar to natural bone for cells, meet the biological requirements of bone tissue engineering, and is expected to become an ideal bioactive scaffold for bone repair.

This application claims priority to CN 201910725714.1, filed Aug. 7, 2019, the contents of which application are incorporated herein by reference in their entireties for all purposes.

### FIELD OF INVENTION

The present invention relates to the field of functional materials for medical use, and particularly to a biomimetic bone composite material, a preparation method and uses thereof.

### BACKGROUD OF THE INVENTION

Natural bone is an organic-inorganic nanocomposite including approximately of 65 wt % of inorganic minerals, 25 wt % of organic matrix and 10 wt % of water (Lowenstam H A, Weiner S. On biomineralization [M]). Oxford University Press on Demand, 1989). The nHA/Collagen composites formed with inorganic nano-hydroxyapatite (nHA) crystallites deposited and aligned parallel to the c-axis of collagen fibril endow natural bone with unique hierarchical structure, excellent biological and mechanical properties. The chemical composition, micro/nanostructure and construction of nHA/Collagen composites all play critical regulating roles in their biological, mechanical and other properties. Therefore, it has been a big challenge to make progress on natural-like bone synthetic materials.

The key factors and mechanisms governing the mineralization process have always been a focus in biomimetic mineralization research. A thorough understanding of the mineralization process can help investigators better imitate nature and design a more biomimetic advanced functional biomaterials in structure and performance. It is generally believed that non-collagenous proteins (NCPs), including osteopontin, osteonectin, osteocalcin, bone sialoprotein, fetuin, dentin matrix protein 1 (DMP1), and non-collagen protein substitutes like polyaspartic acid (PASP), play a crucial regulatory role in the nucleation and growth of HA and bone formation. The critical role of non-collagenous proteins in regulating the nucleation of HA is due to their high acidic amino acid domains which can bind calcium ions and collagen functional groups with high affinity. In addition, the key role of collagen fibril in nucleation has attracted more and more attention. Collagen has functional groups that can interact with calcium ions and phosphate ions of HA, Type I collagen also contains many charged amino acid clusters, and there are many strongly charged sites in fibrils, which is helpful for the infiltration of amorphous calcium phosphate (ACP) into the collagen fibrils.

At present, the study of biomineralization has been mainly focused on organic macromolecule materials like non-collagenous proteins, collagenous extracellular matrixes and their interactions with HA. In particular, a potentially important but often neglected factor is the effect of inorganic trace elements such as silicon on the biomineralization process of collagen. From the perspective of biomimetic, the inorganic components in human skeleton are not only HA, but also have some other inorganic trace elements, such as silicon, strontium, zinc, magnesium and carbonate, which play an important role in the dynamic reorganization of bone. The effect of Si on bone mineralization, as reported by Carlisle in Science as early as 1970, was that the chickens fed with diet contains silicon had much stronger bones than the chickens fed with conventional diet (Carlisle E M. Silicon: a possible factor in bone calcification [J]. Science, 1970, 167(3916): 279-280). The promotion effect of silicon on osteogenesis has been extensively reported both in vivo and in vitro. Currently, four possible mechanisms are widely recognized, including increased dissolution rate, grain size refinement caused by the replacement process, changes in surface charge and surface protein or morphology, but the exact mechanisms remain to be elucidated and even under ongoing argument. (Bohner M. Silicon-substituted calcium phosphates—a critical view [J]. Biomaterials, 2009, 30(32): 6403-6406.) So far, most of studies have focused on the effect of silicon on the cell responses of hydroxyapatite and bioglass. However, the potential effect and mechanism of inorganic trace element Si on the mineralization of collagen fibril have not been reported.

### SUMMARY OF THE INVENTION

The objective of the present invention is to provide a biomimetic bone composite material, a preparation method and uses thereof.

The present invention provides a biomimetic bone composite material, which is prepared from the raw material containing of the following components: gelatin and/or collagen, hydroxyapatite and silicon source.

Further, the weight ratio of each component of the raw material is measured as follows: 5˜35 parts of gelatin and/or collagen, 0.2˜30 parts of hydroxyapatite and 0.001-5 parts of silicon source, the silicon source is measured by the weight of silicon.

Preferably, the weight ratio of each component in the raw material is measured as follows: 10.4˜15.6 parts of gelatin and/or collagen, 1.6˜2.4 parts of hydroxyapatite and 0.012˜0.0192 parts of silicon source, the silicon source is measured by the weight of silicon.

Further preferably, the weight ratio of each component in the raw material is measured as follows: 13 parts of gelatin and/or collagen, 2 parts of hydroxyapatite, 0.016 parts of silicon source, the silicon source is measured by the weight of silicon.

Further, the composite material is electrospun fibrous membrane.

Further, the composite material is prepared by the following method:

a. mixing the hydroxyapatite and the silicon source in water and stirring them thoroughly, then removing water to get the silicon-doped hydroxyapatite for standby; and

b. mixing the silicon-doped hydroxyapatite and the gelatin and/or collagen uniformly.

Further, the silicon-doped hydroxyapatite in step a is synthesized by the following method:

a1. adding Ca(NO₃)₂ and the silicon source into water and stirring them thoroughly;

a2. adding (NH₄)₂ HPO₄ aqueous solution into the mixed solution of Ca(NO₃)₂ and silicon source in stirring, at the same time, maintaining the pH value of the solution at 10±1 by adding a pH regulator, stirring the mixture thoroughly and aging to obtain the precursor solution of silicon-doped hydroxyapatite;

a3. carrying out liquid-solid separation of the precursor solution of silicon-doped hydroxyapatite.

Further, the composite material conforms to at least one of the following:

stirring thoroughly at 60˜80° C. in step a1;

preferably, stirring thoroughly at 70° C. in step a1;

in step a1, the mass to volume ratio of Ca(NO₃)₂ to water is (230˜240):500, g:ml;

in step a2, the concentration of (NH₄)₂ HPO₄ aqueous solution is 70˜80 g/250 mL;

in step a2, adding the (NH₄)₂ HPO₄aqueous solution into the mixed solution of Ca(NO₃)₂ and silicon source in stirring at the speed of 3˜7 ml/min;

the pH regulator in step a2 is ammonia;

the stirring time of the mixture in step a2 is 1-4 hours;

preferably, the stirring time of the mixture in step a2 is 1 hour;

aging at 60˜80° C. in step a2;

preferably, aging at 70° C. in step a2;

the aging time in step a2 is 12˜48 hours;

preferably, the aging time in step a2 is 24 hours;

the drying in step a3 adopts a freeze-drying method;

preferably, in step a3, freeze-drying for 2˜7 days;

preferably, in step a3, freeze-drying for 4 days.

Further, in step b, mixing the silicon-doped hydroxyapatite with the gelatin and/or collagen uniformly by electrospinning.

Preferably, the electrospinning comprises the following steps:

b1. evenly dispersing the silicon-doped hydroxyapatite and the gelatin and/or collagen in water to prepare the homogeneous electrospinning solution;

b2. taking the electrospinning solution for spinning.

Further, in step b1, firstly adding the gelatin and/or collagen into water, stirring and dissolving to get solution A, then adding the silicon-doped hydroxyapatite into solution A and mixing homogeneously to obtain the electrospinning solution.

Further, step b1 conforms to at least one of the following:

adding the gelatin and/or collagen into water according to the mass to volume ratio of (10-15):100, g:ml;

stirring at 35˜70° C. to get solution A;

stirring at the speed of 600 r/min to get solution A;

stirring for 2 hours to get solution A;

adding the silicon-doped hydroxyapatite into solution A according to the mass to volume ratio of (1-5):100, g:ml;

stirring at 35˜70° C. to obtain the electrospinning solution;

stirring at the speed of 600 r/min to obtain the electrospinning solution;

stirring for 2 hours to obtain the electrospinning solution.

Further, step b2 conforms to at least one of the following:

taking the electrospinning solution with a syringe for spinning;

putting a stirrer into the syringe and stirring during spinning;

the stirring rate of stirrers in the syringe is 400-600 r/min;

the inner diameter and outer diameter of the electrospinning needle are 0.51 mm and 0.81 mm respectively;

the spray rate of the electrospinning solution is 0.1˜0.5 ml/h;

the distance between the receiving plate and tip of the needle is 7˜20 cm;

the ambient temperature is 30˜40° C. during the electrospinning;

the positive voltage of the electrospinning needle is 10˜30 kV;

preferably, the positive voltage of the electrospinning needle is 15 kV;

the negative electrode of the power supply is connected to the receiving plate wrapped with aluminum foil.

Further, the method for preparing the composite material also includes the step of crosslinking, wherein the ethanol aqueous solution of the mixture of EDC(N-(3-dimethylaminopropyl)-N′-ethylcarbodiimide hydrochloride) and NHS (N-hydroxysuccinimide) is used for crosslinking.

Further, the step of crosslinking conforms to at least one of the following:

the concentration of the ethanol aqueous solution is 80˜95% v/v;

preferably, the concentration of the ethanol aqueous solution is 90% v/v;

the concentration of EDC is 70˜80 mM;

preferably, the concentration of EDC is 75 mM;

the concentration of NHS is 25˜35 mM;

preferably, the concentration of NHS is 30 mM;

crosslinking at 2˜8° C.;

preferably, crosslinking at 4° C.;

crosslinking for 8˜24 hours;

preferably, crosslinking for 12 hours.

The optimum concentration of the ethanol aqueous solution is around 80˜95% v/v. If the concentration of the ethanol aqueous solution is higher than 95% v/v, it is not conducive to the dissolution of EDC/NHS. If the concentration of the ethanol aqueous solution is lower than 80% v/v, the gelatin absorbs too much water and it's not conducive to maintaining the fibrous structure of gelatin.

Further, the silicon source is a silicon-containing compound capable of hydrolysis.

preferably, the silicon source is a silane coupling agent;

preferably, the silicon source is tetraethyl orthosilicate (TEOS).

The present invention provides a method for preparing the composite material: evenly mixing the hydroxyapatite, silicon source, and gelatin and/or collagen of each weight ratio to obtain the composite material.

The invention provides a use of the composite material in the preparation of bone repair material.

The invention provides a use of silicon as an additive to accelerate bone mineralization in the preparation of biomimetic bone material.

Further, the biomimetic bone material is mainly prepared from gelatin and/or collagen and hydroxyapatite.

Further, the biomimetic bone material is electrospun fibrous membrane.

In general, the hydroxyapatite in the present invention is synthesized by the reaction of calcium nitrate and diammonium hydrogen phosphate under alkaline conditions. During the synthesis process, stirring the solution continuously and finally obtain the slurry, namely the hydroxyapatite suspension. The hydroxyapatite in the suspension is precipitated after aging, then separating the solid and liquid phases and collecting the solid phase to obtain hydroxyapatite.

The synthesis method of silicon-doped hydroxyapatite is basically same as that of hydroxyapatite. The silicon source is added at the beginning of the synthesis process so that the silicon can be incorporated into hydroxyapatite, then the silicon-doped hydroxyapatite is obtained.

Aging can improve the purity and thermal stability of silicon-doped hydroxyapatite; otherwise, the impurity phase of CaO will appear in silicon-doped hydroxyapatite.

The present invention provides a method for repairing bone, which includes applying the composite material to patients in need.

The present invention provides a biomimetic bone composite material with the following beneficial effects:

1. The composite material according to the present invention can accelerate the deposition of calcium ion in a shorter time, thereby enhancing the bone regeneration. This invention demonstrates significant advantages in accelerating the bone mineralization process. In addition, the present invention clearly indicates that the presence of inorganic trace element like silicon has striking influence on the mineralization process, which provides a better reference for the design and optimization of bone repair materials.

2. The composite material of the present invention is doped with trace element silicon, which can simulate the composition of human bone better and promote the bone formation at the surrounding areas.

3. Most of the mineralized products are generated within the fibers and aligned well along the c-axis of the fibers, which have better biomimetic simulating of the bone structure.

4. The optimized ratio of raw material of the present invention is as follows: gelatin and/or collagen of 8-20 wt %, hydroxyapatite of 0.5-20 wt %, sources (salts) of silicon of 0.001-1 wt %. Either the physical and chemical properties of this composite material or the bioactivity and biocompatibility can be modified or improved by adjusting the amount of hydroxyapatite within this range.

5. The present invention preferably uses gelatin to prepare the composite material, which is widely available, inexpensive and has good biocompatibility.

6. The present invention preferably adopts the electrospinning method to prepare the composite material, which has the advantages of simple operation, low cost and suitable for industrial production. The nanofibrous membrane prepared by electrospinning exhibits high porosity and fibrous structure, which is more close to that of the natural extracellular matrix and promote cell adhesion and proliferation.

7. No toxic reagent is involved in the preparation process of the present composite material, thus avoiding the toxic side effects of residues.

### BRIEF DESCRIPTION OF THE DRAWINGS

FIG. 1 shows the scanning electron microscope (SEM) micrographs of electrospun fibersin each group in test example 1;

FIG. 2 shows the SEM micrographs of electrospun fibers in each group after 1 day of mineralization in test example 1;

FIG. 3 shows the SEM micrographs of electrospun fibers in each group after 4 days of mineralization in test example 1;

FIG. 4 shows the SEM micrographs of electrospun fibers in each group after 7 days of mineralization in test example 1;

FIG. 5 shows the macroscopic SEM micrographs of different electrospun fibers after 4 days of mineralization in test example 1;

FIG. 6 shows the FTIR spectrum of electrospun fibers in each group after 1 day of mineralization in test example 1;

FIG. 7 shows the TG/DTG curves of electrospun fibers in each group in test example 1;

FIGS. 8A and 8B show the XPS of electrospun fibers in each group before and after 1 day of mineralization in test example 1;

FIG. 9 shows the XRD patterns of electrospun fibers in each group after 1 day of mineralization in test example 1;

FIG. 10 shows the TEM micrographs of different electrospun fibers after 1 day of mineralization in test example 1;

FIG. 11 shows the TEM micrographs and corresponding SAED patterns of the gelatin-SiHA fibers after mineralization for 1 day and 7 days respectively in test example 1;

FIG. 12 shows the morphology of gelatin electrospun fibers at electrospinning distance of 8 cm according to comparison example 2;

FIG. 13 shows the morphology of electrospun fibers with gelatin concentration of 25% according to comparison example 2; and

FIG. 14 shows the morphology of gelatin electrospun fibers at applied voltage of 25 KV according to comparison example 2.

The patent or application file contains at least one drawing executed in color. Copies of this patent or patent application publication with color drawing(s) will be provided by the Office upon request and payment of the necessary fee.

### DETAILED DESCRIPTION OF PREFERRED EMBODIMENTS

The present invention provides a biomimetic bone composite material which is prepared from the raw material containing the following components: gelatin and/or collagen, hydroxyapatite and a silicon source.

From the biomimetic perspective, we think that as the main inorganic and organic component of bone, nHA and collagen composite material have a great influence on the biological and mechanical properties of bone and other related functions, and there might exist potential synergistic effect from them. While it has been widely reported that the presence of Si and other osteogenesis related trace elements may have a direct influence on nHA, the likely indirect influence of Si on collagen might have been overlooked. The synergistic effect of Si and collagen and the mineralization of collagen may not only have influence but also likely play a key role in directing such process. We find that the incorporation of silicon has a decisive effect on the mineralization process of the nHA-collagen electrospinning composite. The presence of silicon not only significantly promotes the mineralization process of collagen, but also drastically affects the morphology of mineralized crystals. The mineralization of nHA-collagen sample shows the traditional crystal morphology of spherulitic clusters, while the mineralization of nSiHA-collagen sample shows different morphology of mineralized crystal, in a homogeneous nucleation manner.

This kind of mineralization process occurs within collagen fibril and it is mineralized deposition assembled along the c-axis of collagen fibril, renders a highly biomimetic micro/nano-structure which is closer to that of the natural bone. Therefore, the highly biomimetic composite material with fiber network structure in the present invention is expected to be an ideal active scaffold for bone repair material preparation and clinical application.

Furthermore, the optimized choice of substrate material of the present invention is gelatin. Compared with collagen, gelatin is more widely available. As a natural biopolymer derived from collagen, gelatin retains some excellent physical and chemical properties of collagen, such as good biocompatibility, biodegradability and osteoconduction.

Furthermore, the present invention adopts electrospinning technique to synthesize the composite material. Among different techniques in tissue engineering applications, electrospinning is a simple and efficient technique and the nanofiber prepared thereby has many advantages like uniform diameter, high porosity and large specific area. Therefore, from the perspective of better simulating the composition and structure of bone, the present invention adopts the electrospinning technique to prepare gelatin/SiHA electrospun fibrous membrane.

Furthermore, by controlling the parameters of the electrospinning, the morphology of the composite material can be further optimized to obtain a more uniform and smooth fibrous structure.

Wherein, the concentration of gelatin and/or collagen is (10˜15)g/100 mL, to obtain the uniform and smooth fibers.

The distance between the needle tip and the collector is set to 10˜15 cm, to obtain the uniform and smooth fibers.

The applied positive voltage is set to 10˜20 KV, to obtain the uniform and smooth fibers. After repeated tests, we find that the optimized applied positive voltage is 15 kV to obtain the fibers with the best morphology.

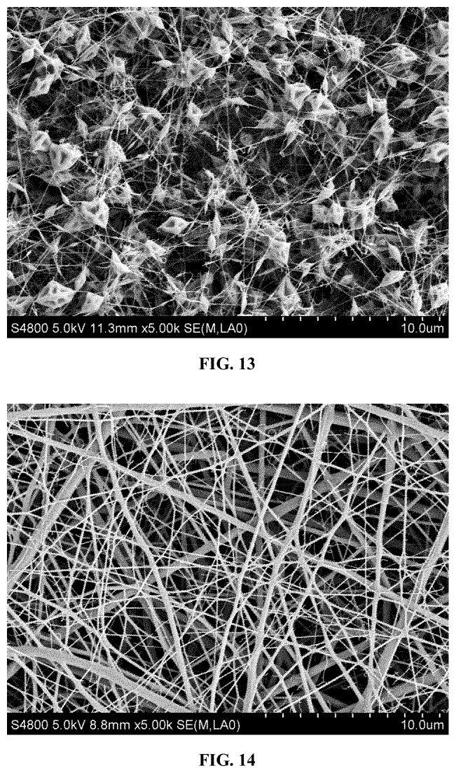

If the parameters of electrospinning are not properly set, the fibers obtained might be not uniform or smooth, or even with the occurrence of some beads (as shown in FIGS. 12˜14). FIGS. 12˜14 show the electrospun fibers obtained by the similar methods as used in embodiment 1. As shown in FIG. 12, during the electrospinning process, the gelatin concentration is 13% (g/100 ml), the distance between the needle tip and the collector is 8 cm, the applied voltage is 15 kV, and the flow rate of solution is 0.3 ml/h. As shown in FIG. 13, during the electrospinning process, the gelatin concentration is 25% (g/100 ml), the distance between the needle tip and the collector is 13 cm, the applied voltage is 15 kV, and the flow rate of solution is 0.3 ml/h. As shown in FIG. 14, during the electrospinning process, the gelatin concentration is 13% (g/100 ml), the distance between the needle tip and the collector is 13 cm, the applied voltage is 25 kV, and the flow rate of solution is 0.3 ml/h.

In summary, the highly biomimetic composite material prepared by the present invention can provide cells with a microenvironment similar to that of natural bone, meet the biological requirements of bone tissue engineering, which is expected to become an ideal active scaffold for bone repair.

The following embodiments further describe the details of the invention. However, these embodiments should not be understood as the limitation of the invention. If the specific techniques or conditions are not shown in these embodiments, it shall be carried out according to the techniques or conditions described in the references in the art or according to the corresponding product specification. If the reagents or equipment used do not illustrate the manufacturers, they are all conventional products that are available on the market.

The gelatin (220 LB 8) used in the following embodiments and examples is purchased from Rousselot (Guangdong) gelatin Co., Ltd., which comes from calf bone and is prepared by alkali method.

### Embodiment 1: Preparation of the Composite Material of the Invention

(1) Synthesis of SiHA

SiHA is prepared through wet chemical method. The specific steps are as follows:

A. First, 118.1 g of (CaNO₃)₂ and 3.1 g of tetraethyl orthosilicate (TEOS) are measured by an electronic balance. Then they are added into 250 ml of deionized water and stirred uniformly in a water bath at 70° C., to make (CaNO₃)₂ dissolved and TEOS further hydrolyzed.

B. Add 37.7 g of (NH₄)₂HPO₄ into 125 ml of deionized water, keep stirring the mixture for more than 30 min with magnetic stirrer to make it completely dissolved.

C. The dissolved (NH₄)₂ HPO₄ solution is pumped into the stirring (CaNO₃)₂ and TEOS solution at the speed of 4× (5 ml/min) by an electronic peristaltic pump. During this process, drop the ammonia into the solution to adjust and keep the pH value to 10±0.2.

D. After completing the addition of (NH₄)₂ HPO₄ solution, keep the mixed solution stirring for 1 hour until the reaction is completed. Then age the mixed solution for 24 hours in the water bath at 70° C. to obtain the SiHA precursor. According to the amount of raw materials, the silicon element doped in the hydroxyapatite is about 0.8% w/w.

E. Put the precursor solution into a beaker after vacuum filtration, then freeze-dry it for 4 days and grind it into powders.

(2) Preparation of the Electrospinning Solution

The electrospinning solution in this invention is made up from the following components by weight: 13 parts of gelatin, 2 parts of SiHA, 100 parts of distilled water. The solution is stirred and heated at the same time to obtain a homogeneous electrospinning solution. The specific steps are as follows: add the gelatin into the distilled water according to the mass volume ratio of 13:100 (g:ml), then heat it at temperature around 35-70° C. and magnetically stir the solution for 2 hours at the speed of 600 r/min for complete dissolve to get solution A; then add the SiHA into solution A according to the mass volume ratio of 2:100 (g:ml). Next, the mixed solution is heated at temperature around 35˜70° C. and magnetically stirred for 2 hours at the speed of 600 r/min to get the electrospinning solution.

(3) Preparation of the Electrospun Fibrous Membrane

The electrospinning solution prepared in step (2) is loaded in a 10 ml syringe with magnetic stirrer therein to keep stirring at a speed of 400-600 r/min so as to prevent the sedimentation of SiHA. The syringe is connected with a stainless steel needle (0.51 mm inner diameter (ID) and 0.81 mm outer diameter (OD)), the positive voltage applied to the needle is 15 kV, and the negative electrode of power supply is connected to the collector wrapped with aluminum foil. The flow rate of the solution is controlled at 0.3 ml/h, the distance between the needle tip and the collector is 13 cm, and the ambient temperature is 35° C. Turn on the high voltage power supply for electrospinning to obtain the fibrous membrane.

(4) Crosslinking of the Electrospun Fibrous Membrane

The membrane obtained in step (3) is cut into a square sample with 1×1 cm². Then use the ethanol aqueous solution with EDC of 75 mM and NHS of 30 mM to crosslink the sample at 4° C. for 12 hours. The crosslinked sample is then washed with distilled water for three times to obtain the fibrous membrane.

### Comparison Example 1: Preparation of the Gelatin/Hydroxyapatite (HA) Electrospun Membrane

(1) Synthesize of HA

HA is prepared through wet chemical method. The specific steps are as follows:

A. First, 119.3 g of (CaNO₃)₂ is measured by an electronic balance. Then it is added into 250 ml of deionized water and stirred uniformly in a water bath at 70° C. to make (CaNO₃)₂ dissolved.

B. Add 40.2 g of (NH₄)₂ HPO₄ into 125 ml of deionized water, keep stirring the mixture for more than 30 min with a magnetic stirrer to make it completely dissolved.

C. The dissolved (NH₄)₂ HPO₄ solution is pumped into the stirring (CaNO₃)₂ solution at the speed of 5 ml/min by an electronic peristaltic pump. During this process, drop the ammonia into the solution to adjust and keep the pH value to 10±0.2.

D. After completing the addition of (NH₄)₂ HPO₄ solution, keep the mixed solution stirring for 1 hour until the reaction is completed. Then age the mixed solution for 24 hours in the water bath at 70° C. and obtain the HA precursor.

E. Put the precursor solution into a beaker after vacuum filtration, then freeze-dry it for 4 days and grind it into powders.

(2) Preparation of the Electrospinning Solution

The electrospinning solution in this invention is made up from the following components by weight: 13 parts of gelatin, 2 parts of HA, 100 parts of distilled water. The solution is stirred and heated at the same time to obtain a homogeneous electrospinning solution. The specific steps are as follows: add the gelatin into the distilled water according to the mass volume ratio of 13:100 (g:ml), then heat it at temperature around 35˜70° C. and magnetically stir the solution for 2 hours at the speed of 600 r/min for complete dissolve to get solution A; then add the HA into solution A according to the mass volume ratio of 2:100 (g:ml). The mixed solution is heated at temperature around 35-70° C. and magnetically stirred for 2 hours at the speed of 600 r/min to get the electrospinning solution.

(3) Preparation of the Electrospun Fibrous Membrane

For the electrospinning process, the electrospinning solution prepared in step (2) is loaded in a 10 ml syringe with magnetic stirrer therein to keep stirring at speed of 400-600 r/min so as to prevent the sedimentation of HA. The syringe is connected with a stainless steel needle (0.51 mm inner diameter (ID) and 0.81 mm outer diameter (OD)). The positive voltage applied to the needle is 15 kV, and the negative electrode of power supply is connected to the collector wrapped with aluminum foil. The flow rate of the solution is controlled at 0.3 ml/h, the distance between the needle tip and the collector is 13 cm, and the ambient temperature is 35° C. Turn on the high voltage power supply for electrospinning to obtain the fibrous membrane.

(4) Crosslinking of Electrospun Fibrous Membrane

The membrane obtained in step (3) is cut into a square sample with 1×1 cm².

Then use ethanol aqueous solution with EDC of 75 mM and NHS of 30 mM to crosslink the sample at 4° C. for 12 hours. The crosslinked sample is then washed with distilled water for three times to obtain the fibrous membrane.

### Comparison Example 2: Preparation of the Gelatin Electrospun Membrane

(1) Preparation of the Electrospinning Solution

The electrospinning solution in this invention is made up from the following components by weight: 13 parts of gelatin, 100 parts of distilled water. The solution is stirred and heated at the same time to obtain a homogeneous electrospinning solution. The specific steps are as follows: add the gelatin into the distilled water according to the mass volume ratio of 13:100 (g:ml), then heat it at temperature around 35˜70° C. and magnetically stir the solution for 2 hours at the speed of 600 r/min for complete dissolve to get the electrospinning solution.

(2) Preparation of the Electrospun Fibrous Membrane

For the electrospinning process, the electrospinning solution prepared in step (1) is loaded in a 10 ml syringe. The syringe is connected with a stainless steel needle (0.51 mm inner diameter (ID) and 0.81 mm outer diameter (OD)), the positive voltage applied to the needle is 15 kV, and the negative electrode of power supply is connected to the collector wrapped with aluminum foil. The flow rate of the solution is controlled at 0.3 ml/h, the distance between the needle tip and the collector is 13 cm, and the ambient temperature is 35° C. Turn on the high voltage power supply for electrospinning to obtain the fibrous membrane.

(3) Crosslinking of the Electrospun Fibrous Membrane

The membrane obtained in step (2) is cut into a square sample 1×1 cm². Then use ethanol aqueous solution with EDC of 75 mM and NHS of 30 mM to crosslink the sample at 4° C. for 12 hours. The crosslinked sample is then washed with distilled water for three times to obtain the fibrous membrane.

The beneficial effects of the present invention are illustrated by the following test examples.

### Test Example 1 The Mineralization Test of the Composite Material

1. Preparation of the Simulated Body Fluid (SBF)

(1) Prepare three 100 ml beakers, stirrers, 1000 ml beaker, 1000 ml volumetric flask and 1000 ml crack free plastic cup and soak them in the acid cylinder overnight, rinse the above containers by ultrapure water before use.

(2) 6.118 g of Tris-hydroxymethyl aminomethane ((HOCH ₂)₃CNH₂) (Tris) is measured and added into a 100 ml beaker, add ultrapure water and stir them until it is completely dissolved.

(3) Add 5 ml of hydrochloric acid (HCl) into 45 ml of ultrapure water and mix them uniformly.

(4) Add stirrers into a 1000 ml beaker and put 700 ml of ultrapure water therein. Set the beaker in a water bath on the magnetic stirrer and cover it with a plastic wrap. Heat the water in the beaker to 36.5±1.5° C. under stirring.

(5) Dissolve the reagents into the solution at 36.5±1.5° C. one by one in the order given in table 1.

(6) Insert an electrode of the pH meter into the solution. Just before dissolving the Tris, the pH value of the solution should be 2.0±1.0.

(7) With the solution temperature between 35 and 38° C., preferably to 36.5±0.5° C., add the reagent Tris into the solution slowly and take careful note of the pH change of the solution at the same time. Add a small amount of Tris gradually when the pH value of the solution becomes 7.30±0.05, and make sure that the temperature of the solution is maintained at 36.5±0.5° C. With the solution at 36.5±0.5° C., add more Tris to raise the pH value to under 7.45. When the pH value has risen to 7.45±0.01, stop adding Tris, then drop HCl by a pipettor to lower the pH value to 7.42. Then add the remaining Tris little by little until the pH value has risen to 7.45±0.01. If any Tris remains, add the HCl and Tris alternately into the solution. Repeat this process until the whole amount of Tris is dissolved and keep the pH value within the range of 7.42-7.45 during dissolve process. After dissolving the whole amount of Tris, adjust the temperature of the solution to 36.5±0.2° C. Adjust the pH of the solution by dropping HCl little by little at a pH value of 7.42±0.01 at 36.5±0.2° C. and then finally adjust the pH value to 7.40 exactly at 36.5° C.

(8) Remove the electrode of the pH meter form the solution, rinse it with ultrapure water and add the washings into the SBF.

(9) Pour the prepared SBF from the beaker into a 1000 ml volumetric flask. Rinse the surface of the beaker with ultrapure water and add the washings into the flask for several times, add the ultrapure water up to the marked line.

(10) The prepared SBF is preserved in a crack free plastic cup with a lid put on tightly and kept at 4° C. in a refrigerator.

TABLE 1
preparation of SBF

| Oder | Reagent | Amount (g) |
|---|---|---|
| 1 | NaCl | 8.035 |
| 2 | NaHCO₃ | 0.355 |
| 3 | KCl | 0.225 |
| 4 | K₂HPO₄•3H₂O | 0.2772 |
| 5 | MgCl₂•6H₂O | 0.311 |
| 6 | HCl | 30 mL |
| 7 | CaCl₂•2H₂O | 0.4656 |
| 8 | Na₂SO₄ | 0.072 |
| 9 | Tris | 6.118 |
| 10 | HCl | 0~5 mL |

2. Mineralization of the Electrospun Membranes

After crosslinking, the samples of embodiment 1, comparison example 1 and comparison example 2 are put into 15 ml centrifuge tubes respectively. Add one piece of membrane and 10 ml of 1.2×SBF in each centrifuge tube, then put them into a 37° C. water bath after being sealed. Take out and wash the samples by ultrapure water after mineralized for 1, 4 and 7 days, put them in a 24-well culture plate.

3. Test Results

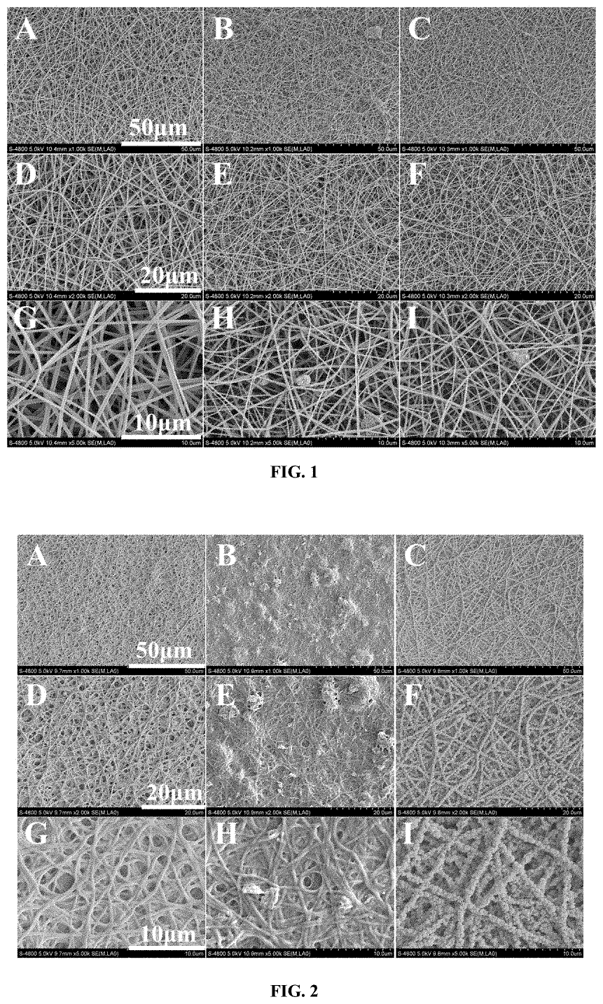

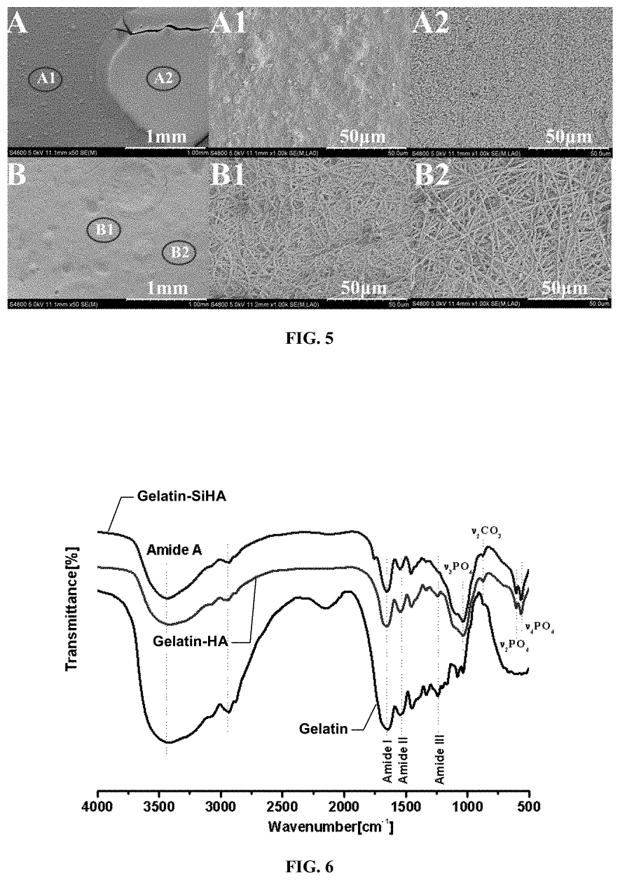

As shown in FIGS. 1-5, the morphologies of the electrospun membranes are observed using SEM. In FIGS. 1-4, A, D, G represent the gelatin electrospun membranes; B, E, H represent the gelatin/HA electrospun membranes; C, F, I represent the gelatin/SiHA electrospun membranes. In FIG. 5, A, A1, A2 represent the gelatin/HA electrospun membranes; B, B1, B2 represent the gelatin/SiHA electrospun membranes.

FIG. 1 shows that well fibrous structures are formed in the gelatin/HA electrospun membranes, the gelatin electrospun membranes and the gelatin/SiHA electrospun membranes prepared respectively in comparison example 1, comparison example 2 and embodiment 1. And the HA particles and SiHA particles are obviously seen on the gelatin/HA electrospun membranes and the gelatin/SiHA electrospun membranes.

As can be seen from FIG. 2 that after 1 day of mineralization in SBF at 37° C., for the gelatin electrospun membrane, the surface is smooth and no minerals are observed thereon; for the gelatin/HA electrospun membrane, there are clusters of CaP minerals locally deposited thereon; while for the gelatin/SiHA electrospun membrane, the CaP minerals spherical deposited along the fiber, and the fibrous' diameter increased obviously after mineralization and the fibrous structure remained intact.

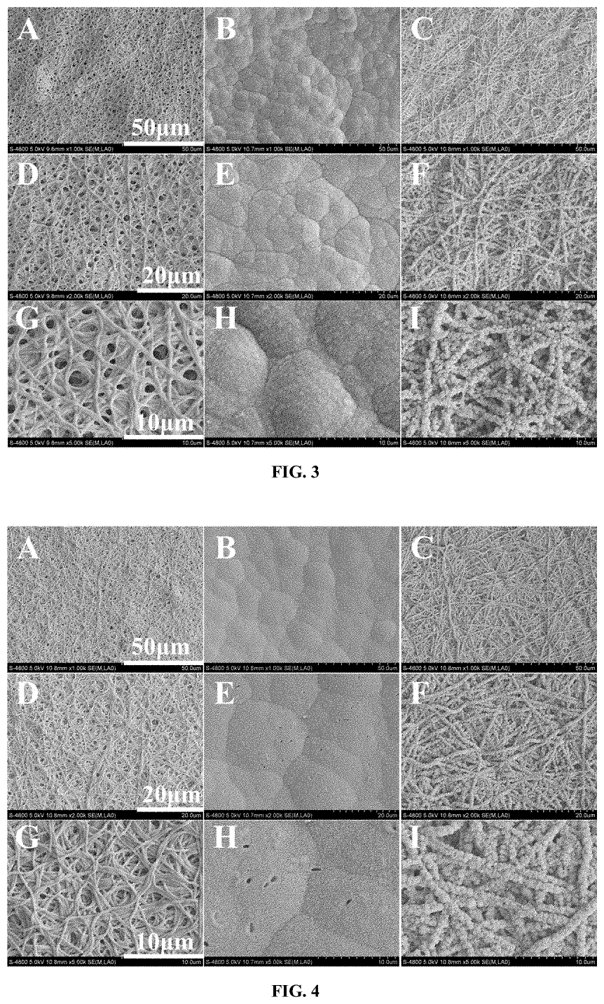

As can be seen from FIGS. 3 and 4 that, as the mineralization time prolonged (4 and 7 days of mineralization respectively), the surface of the gelatin electrospun membrane remains smooth and no minerals are observed on the surface. The CaP crystals on the gelatin/HA electrospun membrane continued to grow and become denser; the membrane loses its fibrous fiber structure characteristics. The gelatin/SiHA membrane exhibits predominant mineralization with CaP crystals assembled along each individual gelatin fibers, and the fibrous structure is maintained during the whole mineralization process, with moderate increase of the fiber diameters.

FIG. 5 shows that the gelatin-SiHA composite nanofibers of embodiment 1 are mineralized uniformly with complete coverage on the whole sample while only parts of the gelatin/HA electrospun sample of comparison example 1 is mineralized.

A FTIR spectrograph (Thermofisher Nicolet 6700) is used to determine the chemical composition of the samples after 1 day of mineralization. The FTIR spectra of the samples are recorded with a range of wave numbers spanning 4000 cm⁻¹-400 cm⁻¹ with averaging over 16 scans. As can be seen from FIG. 6, the characteristic peaks of gelatin can be observed in three groups of samples at 3600-2800 cm⁻¹(amide A), 1740˜1585 cm⁻¹(Amide I), 1560˜1503 cm⁻¹(Amide II), and 1353˜1218 cm⁻¹ (Amide III) respectively. Compared with the spectrum of gelatin, the spectra of gelatin/HA and gelatin/SiHA exhibit additional characteristic peaks at 1035 cm⁻¹, 605 cm⁻¹ and 560 cm⁻¹, attributing to the PO₄³⁻, and another characteristic peak at 873 cm⁻¹ attributing to the CO₃²⁻. These characteristic peaks indicate the existence of mineral phase of hydroxyapatite on gelatin/HA and gelatin/SiHA samples.

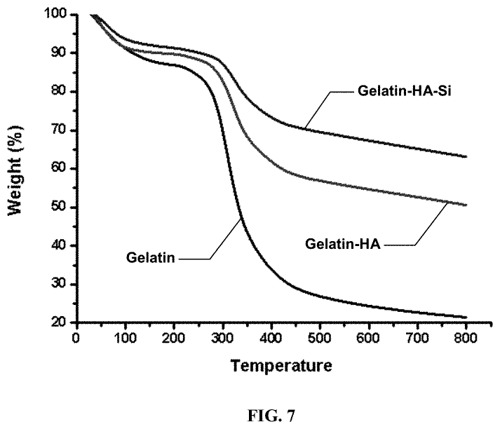

The mineralization degree of three different samples is determined by TGA/DTA (METTLER TOLEDO, Switzerland). After being mineralized for 1 day, these samples are heated from room temperature to 800° C. with a rate of 5° C./min under air to determine the mass of residual mineralized products, as shown in FIG. 7. It can be seen from FIG. 7 that the weight loss of three samples is relatively slow from room temperature to 200° C., which is ascribed to the loss of physisorbed water of composite materials. The weight loss mainly occurs in the range of 200° C.˜490° C., and there are great differences in this temperature range for the weight loss of gelatin, gelatin/HA and gelatin/SiHA are 59.7%, 32.62% and 21.94%, respectively. After the temperature reaches to 490° C., the three different samples all exhibit minor weight loss. Overall, the gelatin, gelatin/HA and gelatin/SiHA mineralized samples show residual mass of around 21.47%, 56.83% and 63.12% respectively. The mineralized products of gelatin/SiHA are more than those of gelatin/HA, and the mineralized products of gelatin/HA are more than those of gelatin.

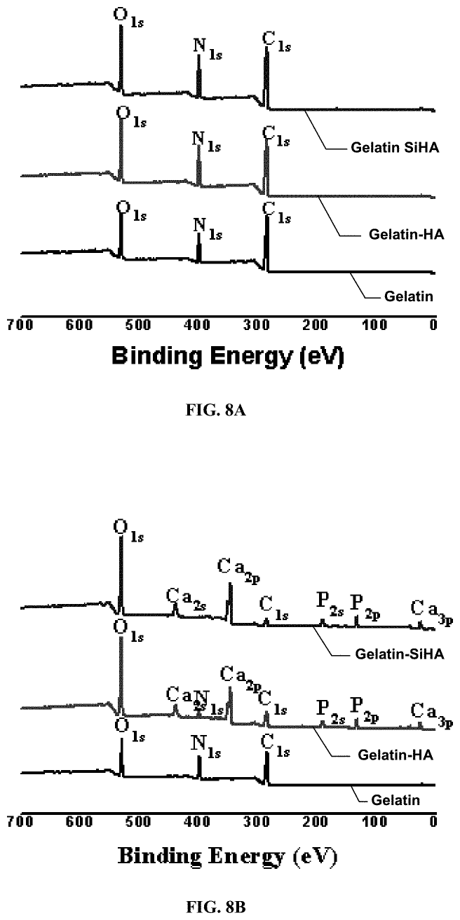

The fiber surface chemistry is examined by X-ray photoelectron spectrometer (ThermoFisher Scientific, USA). The result is shown in FIGS. 8A and 8B. Prior to mineralization, all three samples exhibit similar spectrum, with clearly observed peaks of carbon (C1s), nitrogen (N1s) and oxygen (O1s), major components of gelatin. After 1 day of mineralization, characteristic peaks of Ca and P elements are observed in gelatin/HA sample, and the peaks of C1s and N1s decrease obviously. While the peaks of Ca and P elements are also observed in gelatin/SiHA sample, and the peaks of C1s and N1s decrease more obviously. This further confirms the CaP mineral deposition on the surfaces of gelatin/HA and gelatin/SiHA samples after 1 day of mineralization.

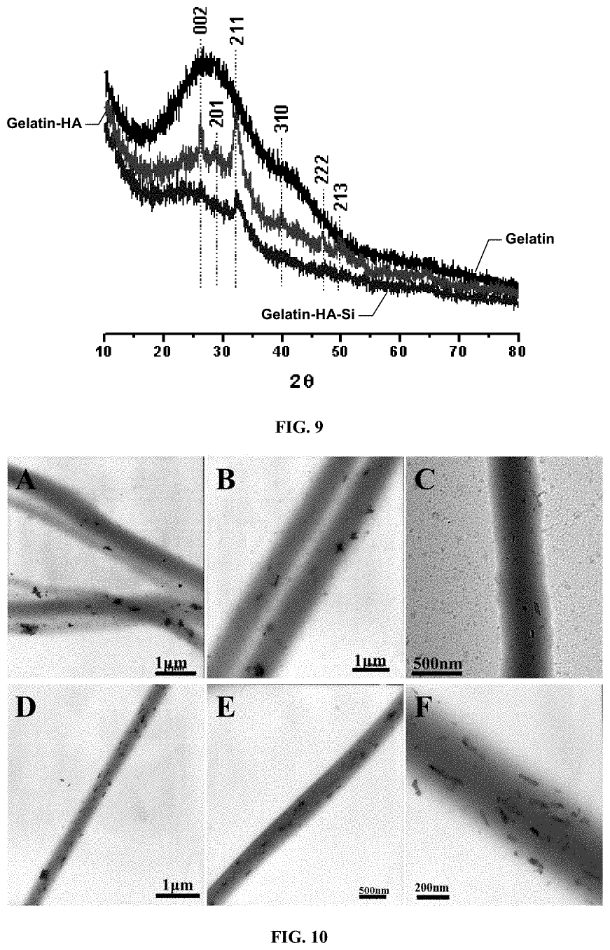

XRD spectra of the mineralized samples are analyzed with EMPYREAN powder X-ray diffractometer. As can be seen form FIG. 9 that no characteristic peak of apatite crystals is observed for the gelatin sample, while the characteristic peaks of apatite crystals are clearly observed for both gelatin/HA and gelatin/SiHA samples at around 2θ=32.1° and 26.5° attributing to the (002) and (211) diffractions of the hydroxyapatite phase(JCPDS#09-432). Minor HA peaks at around 2θ=28.8°, 39.6°, 47° and 49.5° attributing to the (210), (310), (222) and (213) diffractions of the hydroxyapatite phase are also observed in the XRD spectrum of gelatin/HA sample, but not in the gelatin/SiHA sample. The lack of the minor peaks might indicate more confined orientations of mineralized CaP crystals for gelatin/SiHA sample, in comparison with the gelatin/HA one.

TEM analysis has been conducted on the gelatin/HA (FIG. 10 A, B, C) and gelatin/SiHA samples (FIG. 10 D, E, F) after 1 day of mineralization. For gelatin/HA sample, CaP mineralized clusters are sparsely distributed among the collagen region, as well as outside the fiber region, with a crystal size around 40-160 nm, indicating the nucleation occurs in both inside and outside the gelatin fibers for gelatin/HA sample. While significantly higher number of CaP crystals are observed for gelatin/SiHA sample, which are distributed predominantly in the fiber region, and most CaP crystals aligns well along the c-axis of the gelatin fiber with a crystal size around 30-120 nm.

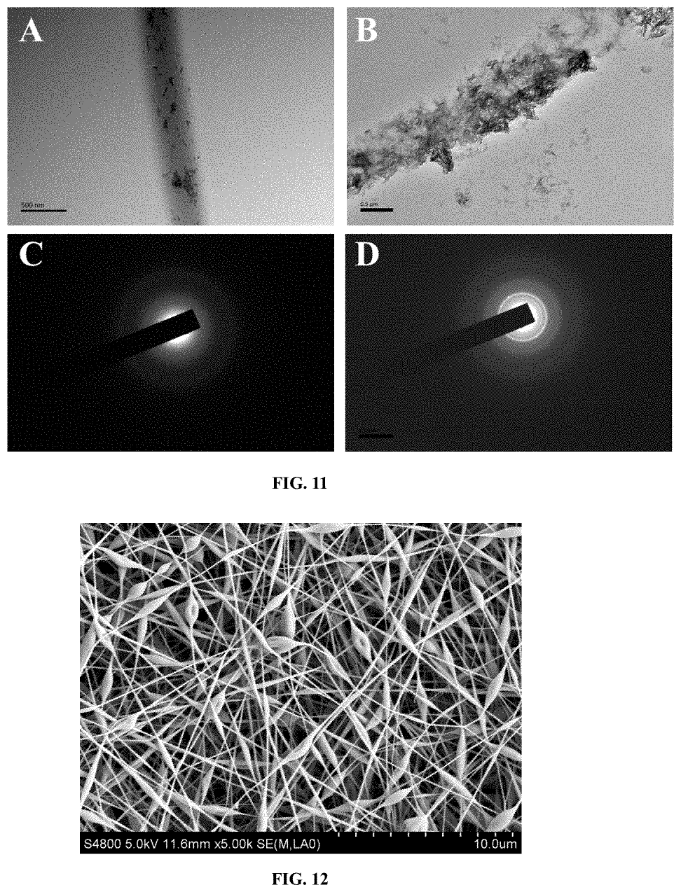

SAED analysis is used to examine the evolution of the CaP phase for gelatin/SiHA sample after 1 d (FIG. 11 A, C) and 7 d mineralization (FIG. 11 B, D). As shown in FIG. 11, sparsely distributed crystal spots are clearly observed with an amorphous background after 1 day of mineralization. After 7 days of mineralization, significant higher number of the crystals are observed along the gelatin fiber in the form of bundles of needle-like crystals, with distinct reflections of the (002) and (211) planes, which is in consistence with the XRD observations.

What is claimed is:

1. A biomimetic bone composite material prepared from a raw material comprising the following components: (a) gelatin and/or collagen; (b) hydroxyapatite; and (c) a silicon source.

2. The composite material according to claim 1, wherein a weight ratio of each component in the raw material is: 5-35 parts of gelatin and/or collagen, 0.2-30 parts of hydroxyapatite and 0.001-5 parts of the silicon source, the silicon source being measured by a weight of silicon.

3. The composite material according to claim 1, wherein a weight ratio of each component in the raw material is: 10.4-15.6 parts of gelatin and/or collagen, 1.6-2.4 parts of hydroxyapatite and 0.0128-0.0192 parts of the silicon source, the silicon source being measured by a weight of silicon.

4. The composite material according to claim 1, wherein a weight ratio of each component in the raw material is: 13 parts of gelatin and/or collagen, 2 parts of hydroxyapatite, 0.016 parts of the silicon source, the silicon source being measured by the weight of silicon.

5. The composite material according to claim 1, which is an electrospun fibrous membrane.

6. A method for preparing the composite material according to claim 1, said method comprising
a. mixing the hydroxyapatite and the silicon source in water with stirring, then removing the water to obtaina silicon-doped hydroxyapatite; and
b. mixing the silicon-doped hydroxyapatite and the gelatin and/or collagen uniformly to obtain the composite material.

7. The method according to claim 6, wherein the silicon-doped hydroxyapatite in step a is synthesized by the following steps:
a1. adding Ca(NO₃)₂ and the silicon source into water and stirring them thoroughly to provide a mixed solution;
a2. adding (NH₄)₂ HPO₄ aqueous solution into the mixed solution of Ca(NO₃)₂ and silicon source with stirring, at the same time, maintaining a pH value of the solution at 10±1 by adding a pH regulator, stirring thoroughly and aging to obtain a precursor solution of silicon-doped hydroxyapatite; and
a3. carrying out liquid-solid separation of the precursor solution of silicon-doped hydroxyapatite, collecting and drying a solid phase.

8. The method according to claim 6, wherein in step b, the mixing is conducted by electrospinning, which comprises the following steps:
b 1. evenly dispersing the silicon-doped hydroxyapatite and the gelatin and/or collagen in water to prepare a homogeneous electrospinning solution; and
b2. electrospinning the homogeneous electrospinning solution.

9. The method according to claim 8, wherein in step b1, firstly adding the gelatin and/or collagen into water, stirring and dissolving to get solution A, then adding the silicon-doped hydroxyapatite into solution A and mixing homogeneously to obtain the electrospinning solution, wherein step b1 further comprises at least one of the following steps:
adding the gelatin and/or collagen into water according to a mass to volume ratio of (10-15):100, g:ml;
stirring at 35-70° C. to get solution A;
stirring at a speed of 600 r/min to get solution A;
stirring for 2 hours to get solution A;
adding the silicon-doped hydroxyapatite into solution A according to the mass to volume ratio of (1-5):100, g:ml;
stirring at 35˜70° C. to obtain the electrospinning solution;
stirring at the speed of 600 r/min to obtain the electrospinning solution; and
stirring for 2 hours to obtain the electrospinning solution.

10. The method according to claim 8, wherein step b2 comprises loading the electrospinning solution in a syringe having an electrospinning needle, and at least one of the following steps:
putting a stirrer into the syringe and stirring during spinning;
providing a stirring rate of the stirrer in the syringe of 400-600 r/min;
providing the electrospinning needle with an inner diameter of 0.51 mm and an outer diameter of 0.81 mm;
spraying the homogeneous electrospinning solution at 0.1-0.5 ml/h;
providing a distance between a receiving plate and a tip of the electrospinning needleof 7-20 cm;
conducting the electrospinning at an ambient temperature of 30-40° C.;
providing the electrospinning needle with a positive voltage of 10-30 kV; and
connecting a negative electrode of a power supply to the receiving plate wrapped with aluminum foil.

11. The method according to claim 10, wherein the positive voltage of the electrospinning needle is 15 kV.

12. The method according to claim 6, further comprising the step of crosslinking using an ethanol aqueous solution of a mixture of EDC and NHS.

13. The composite material according to claim 1, wherein the silicon source is a silicon-containing compound capable of hydrolysis.

14. The composite material according to claim 1, wherein the silicon source is a silane coupling agent.

15. The composite material according to claim 1, wherein the silicon source is tetraethyl orthosilicate.

16. A biomimetic bone composite material prepared by the method according to claim 6.

17. A bone repair material comprising the composite material according to claim 1.

18. A bone repair material comprising the composite material according to claim 16.

19. A method for preparing a biomimetic bone material comprising using silicon as an additive to accelerate bone mineralization.

20. The method according to claim 19, wherein the biomimetic bone material is mainly prepared from gelatin and/or collagen and hydroxyapatite.
